SEQUENCE LISTING (24 sequences)

SEQ ID NO: 1 - protein, 904 aa, Homo sapiens
MRQTLPCIYFWGGLLPFGMLCASSTTKCTVSHEVADCSHLKLTQVPDDLPTNITVLNLTHNQLRRLPAANFTRYSQLTSLDVGFNTISKLEPELCQKLPMLKVLNLQHNELSQLSDKTFAFCTNLTELHLMSNSIQKIKNNPFVKQKNLITLDLSHNGLSSTKLGTQVQLENLQELLLSNNKIQALKSEELDIFANSSLKKLELSSNQIKEFSPGCFHAIGRLFGLFLNNVQLGPSLTEKLCLELANTSIRNLSLSNSQLSTTSNTTFLGLKWTNLTMLDLSYNNLNVVGNDSFAWLPQLEYFFLEYNNIQHLFSHSLHGLFNVRYLNLKRSFTKQSISLASLPKIDDFSFQWLKCLEHLNMEDNDIPGIKSNMFTGLINLKYLSLSNSFTSLRTLTNETFVSLAHSPLHILNLTKNKISKIESDAFSWLGHLEVLDLGLNEIGQELTGQEWRGLENIFEIYLSYNKYLQLTRNSFALVPSLQRLMLRRVALKNVDSSPSPFQPLRNLTILDLSNNNIANINDDMLEGLEKLEILDLQHNNLARLWKHANPGGPIYFLKGLSHLHILNLESNGFDEIPVEVFKDLFELKIIDLGLNNLNTLPASVFNNQVSLKSLNLQKNLITSVEKKVFGPAFRNLTELDMRFNPFDCTCESIAWFVNWINETHTNIPELSSHYLCNTPPHYHGFPVRLFDTSSCKDSAPFELFFMINTSILLIFIFIVLLIHFEGWRISFYWNVSVHRVLGFKEIDRQTEQFEYAAYIIHAYKDKDWVWEHFSSMEKEDQSLKFCLEERDFEAGVFELEAIVNSIKRSRKIIFVITHHLLKDPLCKRFKVHHAVQQAIEQNLDSIILVFLEEIPDYKLNHALCLRRGMFKSHCILNWPVQKERIGAFRHKLQVALGSKNSVH

SEQ ID NO: 2 - DNA, 24 bp, Artificial Sequence
  phosphorothioate backbone
tcgtcgttttgtcgttttgtcgtt

SEQ ID NO: 3 - DNA, 24 bp, Artificial Sequence
  phosphorothioate backbone
tgctgcttttgtgcttttgtgctt

SEQ ID NO: 4 - DNA, 20 bp, Artificial Sequence
  mixed phosphodiester(nt 3-14)/phosphorothioate(nt 1-2,15-20) backbone
gggggacgatcgtcgggggg

SEQ ID NO: 5 - DNA, 20 bp, Artificial Sequence
  mixed phosphodiester(nt 3-14)/phosphorothioate(nt 1-2,15-20) backbone
gggggagcatgctggggggc

SEQ ID NO: 6 - DNA, 25 bp, Artificial Sequence
  phosphodiester backbone
gtcgacaagggattgaacctcgttc

SEQ ID NO: 7 - DNA, 30 bp, Artificial Sequence
  phosphodiester backbone
tacgtacttagatatgtcttcaaaccatac

SEQ ID NO: 8 - DNA, 36 bp, Artificial Sequence
  phosphodiester backbone
ggcttcactttcatagactgcaacggtgcctgaaag

SEQ ID NO: 9 - DNA, 40 bp, Artificial Sequence
  phosphodiester backbone
gctctagattaagatttaccgatgtcgctcactaaggacc

SEQ ID NO: 10 - DNA, 54 bp, Artificial Sequence
  phosphodiester backbone
aaagtgtgtcactgccagtgtgcaaggatagactggacagcagcccgctgctgt

SEQ ID NO: 11 - DNA, 60 bp, Artificial Sequence
  phosphodiester backbone
ctcgagcagaggtctcacacagagacaagcgcatcagtcaacagagatcccttgcgcttc

SEQ ID NO: 12 - DNA, 75 bp, Artificial Sequence
  phosphodiester backbone
gtcgaccacggttctgctacttgttctttgtttttcaccaacaaaatggtatggtttgaagacatatctaagtac

SEQ ID NO: 13 - DNA, 39 bp, Artificial Sequence
  phosphodiester backbone
acaaacataacagtgttggcccttacccataatcaactc

SEQ ID NO: 14 - DNA, 34 bp, Artificial Sequence
  phosphodiester backbone
cataacagtgttggcccttacccataatcaactc

SEQ ID NO: 15 - DNA, 29 bp, Artificial Sequence
  phosphodiester backbone
cagtgttggcccttacccataatcaactc

SEQ ID NO: 16 - DNA, 24 bp, Artificial Sequence
  phosphodiester backbone
ttggcccttacccataatcaactc

SEQ ID NO: 17 - DNA, 19 bp, Artificial Sequence
  phosphodiester backbone
ccttacccataatcaactc

SEQ ID NO: 18 - DNA, 34 bp, Artificial Sequence
  phosphodiester backbone
acaaacataacagtgttggcccttacccataatc

SEQ ID NO: 19 - DNA, 29 bp, Artificial Sequence
  phosphodiester backbone
acaaacataacagtgttggcccttaccca

SEQ ID NO: 20 - DNA, 24 bp, Artificial Sequence
  phosphodiester backbone
acaaacataacagtgttggccctt

SEQ ID NO: 21 - DNA, 19 bp, Artificial Sequence
  phosphodiester backbone
acaaacataacagtgttgg

SEQ ID NO: 22 - DNA, 36 bp, Artificial Sequence
  mixed phosphodiester(nt 1-6,31-36)/phosphorothioate(nt7-30) backbone
ccgccctcgtcgttttgtcgttttgtcgttgggcgg

SEQ ID NO: 23 - DNA, 32 bp, Artificial Sequence
  mixed phosphodiester(nt 1-6,27-32)/phosphorothioate(nt7-26) backbone
ccgcccttttttttttttttttttttgggcgg

SEQ ID NO: 24 - DNA, 32 bp, Artificial Sequence
  mixed phosphodiester(nt 1-6,27-32)/phosphorothioate(nt7-26) backbone
ccgcccaaaaaaaaaaaaaaaaaaaagggcgg

(19) United States
(12) Patent Application Publication
(10) Pub. No.: US 2008/0299138 A1
(43) Pub. Date: Dec. 4, 2008

(54) Toll-Like Receptor 3 Modulators and Uses Thereof

(76) Inventors: Karen E. Duffy, Radnor, PA (US); Cheneparath Tharachaparamba Ranjith-Kumar, Bloomington, IN (US); Jarrat L. Jordan, Radnor, PA (US); Cheng Chia Kao, College Station, TX (US); Robert T. Sarisky, Radnor, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/126,150

(22) Filed: May 23, 2008

Related U.S. Application Data
(60) Provisional application No. 60/940,196, filed on May 25, 2007.

(51) Int. Cl. A61K 39/44; A61K 31/7088; A61K 38/00; C07H 21/04; A61P 11/00; A61P 19/02; A61P 35/04; A61P 9/00; A61P 3/10; A61P 29/00; A61P 25/00

(52) U.S. Cl. 424/178.1; 514/44; 514/2; 536/23.5

(57) ABSTRACT

Modulators of TLR3 activity and their use are disclosed.

### CROSS-REFERENCE TO RELATED APPLICATIONS

This application claims priority to U.S. Provisional Application No. 60/940,196, filed 25 May 2007, the entire contents of which is incorporated herein by reference in its entirety.

### FIELD OF THE INVENTION

This invention relates to oligonucleotide modulators of toll-like receptor 3 (TLR3) activity and their use.

### BACKGROUND OF THE INVENTION

Innate immune receptors are promising targets to regulate the complex cascade of reactions that will lead to cytokine production.⁴ These receptors participate in this process by recognizing pathogen ligands through their molecular signatures and then use several signaling cascades to alter gene expression. The Toll-like receptors are a family of structurally related class I single pass transmembrane proteins that serve as the sentries for pathogen infections.⁵⁻⁷ At least eleven TLRs have been identified in the mammalian genome that can be generally segregated by the pathogen molecules that they recognize, such as highly conserved bacterial proteins, pathogen cell wall components, and pathogen-associated nucleic acids.⁸

There are four nucleic acid-binding TLRs: Toll-like receptors 7 and ⁸, which recognize single-stranded RNAS,⁴⁻⁶ TLR9, which recognizes single-stranded DNA molecules that contain hypomethylated CpG motifs,⁹ and TLR3, which recognizes double-stranded RNAs.¹⁰ In laboratory studies, poly(I:C), a synthetic double-stranded (ds) RNA analog, has served as a model dsRNA and a TLR3 ligand.¹¹ Poly(I:C) is bound by TLR3 especially at lower pHs, perhaps suggesting that TLR3 may bind to dsRNA ligands within the confines of acidic vesicles, a site where TLR3 has been localized.^{12,13} A full-length human TLR3 amino acid sequence is shown in SEQ ID NO: 1.

TLR3 binding to cognate ligands modulates downstream cytokine and chemokine production through the activation of the transcription factor NF-κB, which translocates to the nucleus to modulate gene expression.^{14,15} A role for TLR3 in viral infection has been suggested based on the demonstration that TLR3 knockout mice were unable to mount a full response to cytomegalovirus infection,¹⁶ perhaps by contributing to cytotoxic T cell response after the initial infection.¹⁷

A reporter assay for TLR3 based on NF-κB activation has been established and is commonly used by practitioners in the field.^{14,15} The effects of TLR3 could also be monitored by assessing the amount of cytokines and chemokines produced, such as Interferon-gamma, Interleukin-12, and IL-1α, IP-10, and MIG.¹⁸ TLR3 activation of NF-κB reporter or cytokine production is recognized as “TLR3 activity”.

The types and amounts of cytokine produced by TLR3 activity can dictate the outcome of pathogen infection, and cause a suite of inflammation-associated systems that characterize several diseases, including colitis, asthma, psoriasis, and septic shock.¹⁻³ Further, in necrotic conditions, the release of intracellular content after cellular membrane damage triggers inflammation expression of cytokines, chemokines and other factors to facilitate clearance of dead cell remnants and repair the damage. Necrosis often perpetuates chronic or aberrant inflammatory processes leading to secondary damage or cascade of effects. Thus, a need exists to control cytokine production through down modulation of TLR3 activity.

### BRIEF DESCRIPTION OF THE DRAWINGS

FIG. 1 shows the effect of ODN2006 on TLR3 and TLR9 activity in the presence of poly(I:C).

FIG. 2 shows the effect of ODN2006 concentration on TLR3 activity.

FIG. 3 shows the effect of poly(I:C) on inhibition of TLR3 activity by ODN2006.

FIG. 4 shows the effect of ODN2006 on TLR3 activity after poly(I:C) activation.

FIG. 5 shows the effect of type A and type B oligonucleotides and their controls on TLR3 activity.

FIG. 6 shows the effect of oligonucleotide stability on TLR3 activity.

FIG. 7 shows the effect of phosphodiester oligonucleotides on TLR3 activity.

FIG. 8 shows the effect of oligonucleotide length on TLR3 activity.

FIG. 9 shows interferon-γ (IFNγ) production by human PBMC.

### SUMMARY OF THE INVENTION

One aspect of the invention is a method for down modulating toll-like receptor 3 (TLR3) activity in a mammal comprising administering at least one inhibitory oligonucleotide (iOGN) having TLR3 down modulating activity to the mammal.

Another aspect of the invention is a method of treating or preventing an inflammatory condition comprising administering a therapeutically effective amount of a TLR3 iOGN to a patient in need thereof for a time sufficient to treat or prevent the inflammatory condition.

Another aspect of the invention is a method of treating or preventing a necrotic condition comprising administering a therapeutically effective amount of a TLR3 iOGN to a patient in need thereof for a time sufficient to treat or prevent the necrotic condition.

Another aspect of the invention is a method of treating or preventing an infectious disease comprising administering a therapeutically effective amount of a TLR3 iOGN to a patient in need thereof for a time sufficient to treat or prevent the infectious disease.

Another aspect of the invention is a method of treating or preventing a cardiovascular disease comprising administering a therapeutically effective amount of a TLR3 iOGN to a patient in need thereof for a time sufficient to treat or prevent the cardiovascular disease.

Another aspect of the invention is a method of treating or preventing type I or type II diabetes comprising administering a therapeutically effective amount of a TLR3 iOGN to a patient in need thereof for a time sufficient to treat or prevent the type I or type II diabetes.

Another aspect of the invention is a method of treating or preventing cancer comprising administering a therapeutically effective amount of a TLR3 iOGN to a patient in need thereof for a time sufficient to treat or prevent the cancer.

Another aspect of the invention is a method of treating or preventing rheumatoid disease comprising administering a therapeutically effective amount of a TLR3 iOGN to a patient in need thereof for a time sufficient to treat or prevent the rheumatoid disease.

Another aspect of the invention is a method of treating or preventing pulmonary disease comprising administering a therapeutically effective amount of a TLR3 iOGN to a patient in need thereof for a time sufficient to treat or prevent the pulmonary disease.

Another aspect of the invention is a method of treating or preventing neurological disorders comprising administering a therapeutically effective amount of a TLR3 iOGN to a patient in need thereof for a time sufficient to treat or prevent the neurological disorders.

Another aspect of the invention is an iOGN having the sequence shown in SEQ ID NO: 7, 8, 9, 10, 11, 12, 13, 14, 15, 16, 18, 19, 20, 21, 22 or 23.

### DETAILED DESCRIPTION OF THE INVENTION

All publications, including but not limited to patents and patent applications, cited in this specification are herein incorporated by reference as though fully set forth.

The term “TLR3 inhibitory oligonucleotide (iOGN)” or “iOGN” as used herein refer to or describe a molecule that is capable of, directly or indirectly, substantially reducing or inhibiting TLR3 biological activity or TLR3 receptor activation. These terms are used to refer to the singular and the plural.

The term “in combination with” as used herein means that the described agents can be administered to an animal together in a mixture, concurrently as single agents or sequentially as single agents in any order.

The present invention relates to single-stranded inhibitory oligonucleotide (iOGN) down modulators of TLR3 activity. The modulators of the invention can be oligodeoxyribonucleotides or oligodeoxynucleotides and significantly down modulate the gene expression pattern initiated by human Toll-like Receptor 3 (TLR3) thereby regulating cytokine production. Cytokine secretion is a key intermediate step in the generation of an immune response. The IOGN modulators of the invention are useful for treatment or prevention of pathological disorders characterized by inflammation or necrosis in mammals such as humans.

Published reports on the effects of TLR3 and TLR9 ligand combinations in murine cells show enhanced cytokine responses after stimulation with poly(I:C) and CpG oligodinucleotides (ODN)²³. Unexpectedly, in the present invention, certain ODN were observed to have down-modulatory activity on poly(I:C)-induced TLR3 activation in human cells resulting in decreased cytokine production. These ODN, their derivatives and other oligonucleotides with TLR3 down-modulating activity are hereinafter identified as inhibitory oligonucleotides (iOGN).

The sequences of iOGN molecules that down modulate TLR3 are distinct from those that activate a related Toll-like receptor, TLR9. Further, these iOGN molecules can have mixed phosphodiester and phosphorothioate, or only phosphodiester linkages. Exemplary iOGN sequences are shown in SEQ ID NOs: 2, 3, 4, 5, 7, 8, 9, 10, 11, 12, 13, 14, 15, 16, 18, 19, 20, 21, 22 and 23. Further, the down modulatory effects of iOGN are not affected by the presence of any of TLR1, 2, 4, 5, 6, 7, or 8. It is also contemplated that the iOGN of the invention can comprise modified bases, ribose derivatives and/or other phosphodiester or phosphorothioate linkage derivatives. Modifications include natural phosphoramidites, 2′-oMe, locked nucleic acid (LNA), peptide nucleic acid (PNA), ribonucleic acids (RNA), F-RNA and other modified bases.

The invention further relates to design of iOGN with TLR3 modulating activity. The degree of modulation can be manipulated by the properties of the iOGN molecules, including their length, base sequence, and the degree of modification. The observed structure-activity relationships of the iOGN of the invention can be useful as a platform to design molecules that can influence the outcome of numerous human diseases, with an emphasis on pathological disorders characterized by inflammation or necrosis.

In one embodiment, the present invention provides a method for use of one unmodified iOGN or an unmodified iOGN in combination with one or more unmodified iOGN of different lengths and/or base sequences for down modulating TLR3 activity in a mammal (such as a human) to decrease cytokine and chemokine production stimulated by TLR3.

In another embodiment, the method of the invention provides for the use of at least one iOGN in combination with another non-iOGN modulator of TLR3 activity. The non-iOGN modulator can be an antibody, MIMETIBODY™ construct, or small molecule specific for TLR3 or another TLR receptor. A MIMETIBODY™ construct has the generic formula (I):

(Bp-Lk-(V2)_{y}-Hg—C_{H}2-C_{H}3)₍ₜ₎ (I)

where Bp is a peptide or polypeptide capable of binding a molecule of interest, Lk is a polypeptide or chemical linkage, V2 is a portion of a C-terminus of an immunoglobulin variable region, Hg is at least a portion of an immunoglobulin variable hinge region, C_{H}2 is an immunoglobulin heavy chain C_{H}2 constant region and C_{H}3 is an immunoglobulin heavy chain C_{H}3 constant region, y is 0 or 1, and t is independently an integer of 1 to 10.

In another embodiment, a single or combination of chemically and covalently modified iOGN that can confer desirable properties including, but not limited to, increased stability, increased ability to traverse cells and cell membranes, increased specificity in affecting TLR3 activity, could be used to modulate cytokine and chemokine production by TLR3. The modifications could consist of small molecular moieties or dyes, of which some examples include additions to, or alterations of, the nucleotide base, ribose and the phosphodiester group found in nucleotides. The modifications could also include macromolecules such as proteins, other DNAs, RNAs, and polysaccharides that can be covalently or noncovalently linked to the DNA. The modifications could also include one or more small molecule or macromolecule or a small molecule or macromolecule with several subunits. Further, the modifications could also include esterified or partially esterified phosphonoacetates to improve bioavailability.

In yet another embodiment of the invention, the iOGN is conjugated to a monoclonal antibody, antibody fragment, alternative scaffold such as designed ankyrin repeat proteins (DARPins)^{22, 24}, protein, MIMETIBODY™ construct or peptide specific for TLR3.

In yet another embodiment, the method of the invention provides for the use of at least one iOGN in combination with an anti-inflammatory agent.

In yet another embodiment, the method of the invention provides for the use of at least one iOGN in combination with an anti-microbial agent, including anti-fungal or anti-protist agents.

In yet another embodiment, the method of the invention provides for the use of at least one iOGN in combination with an anti-viral agent.

While not wishing to be bound to any particular theory, it is thought that the iOGN of the invention will act directly on TLR3, perhaps by binding to one or more sites within the TLR3 molecule, or indirectly, perhaps by preventing an accessory protein from contributing to TLR3 function.

iOGN with TLR3 down modulating activity are useful for treatment and prophylaxis of a number of mammalian disease states including, but not limited to, inflammatory conditions, necrotic conditions, infectious diseases, cardiovascular disease, type I diabetes, type II diabetes, cancer, rheumatoid disease, pulmonary disease and neurological disorders.

Exemplary inflammatory conditions include infection-associated inflammation as well as pancreatitis, alopecia areata, atopic dermatitis, autoimmune hepatitis, Bechet's disease, cirrhosis, hepatic fibrosis, Crohn's disease, regional enteritis, inflammatory vitilgo, multiple sclerosis, pemphigus/pemphigoid, primary biliary cirrhosis, psoriasis, scleroderma, sclerosing cholangitis, systemic lupus erythematosus, lupus nephritis, toxic epidermal necrolysis, ulcerative colitis, warts, hypertrophic scarring, keloids and acetaminophen-induced injury.

Exemplary necrotic conditions include acute renal failure.

Exemplary infectious diseases include anthrax, C. Difficile infection, encephalitis/meningitis, endocarditis, Hepatitis C, Influenza/severe acute respiratory syndrome (SARS), pneumonia, sepsis, burn or trauma-related skin indications and systemic inflammatory response syndrome (SIRS).

Exemplary cardiovascular disease includes atherosclerosis, myocardial infarction and stroke.

Exemplary cancers include acute leukemia, breast cancer, chronic leukemia, colorectal cancer, esophageal cancer, gastric cancer, Hodgkins disease, lung cancer, lymphoma, melanoma, multiple myeloma, Non-hodgkin's disease, ovarian cancer, pancreatic cancer, prostrate cancer, sarcoma, renal cell cancer, head and neck cancers and virally-induced cancers.

Exemplary rheumatoid disease includes autoimmune thyroiditis, autoimmune vasculitis, disoid lupus erythematosus, lupus nephritis, osteoarthritis, polychondritis, polymyalgia rheumatica, psoriatic arthritis, rheumatoid arthritis, systemic lupus erythematosus and systemic scleroderma.

Exemplary pulmonary disease includes acute lung injury, acute respiratory distress syndrome (ARDS), acute asthma exacerbations, acute COPD exacerbations, idiopathic pulmonary fibrosis or sarcoid.

Exemplary neurological disorders include stroke, Alzheimer's disease, meningitis, spinal cord injury, trauma, demyelination disorders and pain.

The iOGN useful in the invention can be made by oligonucleotide synthesis techniques well known to those skilled in the art.

The mode of administration for therapeutic or prophylactic use of the iOGN of the invention may be any suitable route that delivers the agent to the host. The ODNs and any combination therapy partners such as small molecules, antibodies, antibody fragments and mimetibodies and pharmaceutical compositions of these agents can be delivered by parenteral administration, i.e., subcutaneously, intramuscularly, intradermally, intravenously or intranasally as well as by topical or aerosol routes for delivery directly to target organs such as the lungs.

The iOGN of the invention may be prepared as pharmaceutical compositions containing an effective amount of the agent as an active ingredient in a pharmaceutically acceptable carrier. An aqueous suspension or solution containing the agent, preferably buffered at physiological pH, in a form ready for injection is preferred. The compositions for parenteral administration will commonly comprise a solution of the binding agent of the invention or a cocktail thereof dissolved in a pharmaceutically acceptable carrier, preferably an aqueous carrier. A variety of aqueous carriers may be employed, e.g., 0.4% saline, 0.3% glycine and the like.

Solutions of these pharmaceutical compositions are sterile and generally free of particulate matter. These solutions may be sterilized by conventional, well-known sterilization techniques (e.g., filtration). The compositions may contain pharmaceutically acceptable auxiliary substances as required to approximate physiological conditions such as pH adjusting and buffering agents, etc. The concentration of the ODNs of the invention in such pharmaceutical formulation can vary widely, i.e., from less than about 0.5%, usually at or at least about 1% to as much as 15 or 20% by weight and will be selected primarily based on fluid volumes, viscosities, etc., according to the particular mode of administration selected.

Thus, a pharmaceutical composition of the invention for intramuscular injection could be prepared to contain 1 mL sterile buffered water, and between about 1 ng to about 100 mg, e.g. about 50 ng to about 30 mg or, more particularly, about 5 mg to about 25 mg of an iOGN of the invention. Similarly, a pharmaceutical composition of the invention for intravenous infusion could be made up to contain about 250 ml of sterile Ringer's solution, and about 1 mg to about 30 mg or, more particularly, about 5 mg to about 25 mg of an ODN of the invention. Actual methods for preparing parenterally administrable compositions are well known or will be apparent to those skilled in the art and are described in more detail in, e.g., “Remington: The Science and Practice of Pharmacy (Formerly Remington's Pharmaceutical Sciences)”, 19th ed., Mack Publishing Company, Easton, Pa. (1995).

The iOGN of the invention, when in a pharmaceutical preparation, can be present in unit dose forms. The appropriate therapeutically effective dose can be determined readily by those of skill in the art. A determined dose may, if necessary, be repeated at appropriate time intervals selected as appropriate by a physician during the treatment period.

The iOGN of the invention can be lyophilized for storage and reconstituted in a suitable carrier prior to use. This technique has been shown to be effective with conventional immunoglobulins and protein preparations and art-known lyophilization and reconstitution techniques can be employed.

The present invention will now be described with reference to the following specific, non-limiting examples.

### EXAMPLE 1

### Determination of Effects of Single-Stranded DNA on Cytokine/Chemokine Production by Human Cells in Culture

Human embryonic kidney cells (HEK 293T) were harvested from an actively growing culture and plated in CoStar White 96-well plates at 4.4×10⁴/well for transfection. When the cells were ˜85 to 90% confluent, they were transfected with a mixture of the Lipofectamine 2000 (Invitrogen Inc., San Diego, Calif.) and plasmids pNF-κB-Luc (Stratagene) or pNiFty-Luc (Invivogen, San Diego, Calif.), pUNO-huTLR3 (Invivogen), and phRL-TK (Promega Corp., Madison, Wis.) that, respectively, code for the firefly luciferase reporter, full-length wild-type TLR3, and the Renilla luciferase transfection control. The cells were allowed to incubate for 24 h to allow expression from the plasmids. Poly(I:C) (2.5 μg/mL) and/or the single-stranded modified DNA known as ODN2006 was then added to appropriate sets of transfected cells to effect TLR3-dependent NF-κB activity. Poly(I:C) was purchased from GE Amersham and reconstituted in PBS while heating at 50° C. ODN2006 was obtained from Invivogen. After another 24 h incubation, the cells were harvested using the Dual Glo Luciferase Assay System reagents (Promega Inc., Madison Wis.). Luminescence was measured using a FLUOstar OPTIMA Plate Reader (BMG Labtech, Inc). Data is presented as either a luciferase ratio, which is derived by dividing the NF-κB firefly relative light units (RLUs) by the control Renilla RLUs, or a fold induction, in which all treatment group luciferase ratios are divided by the unstimulated TLR3-transfected cell luciferase ratio.

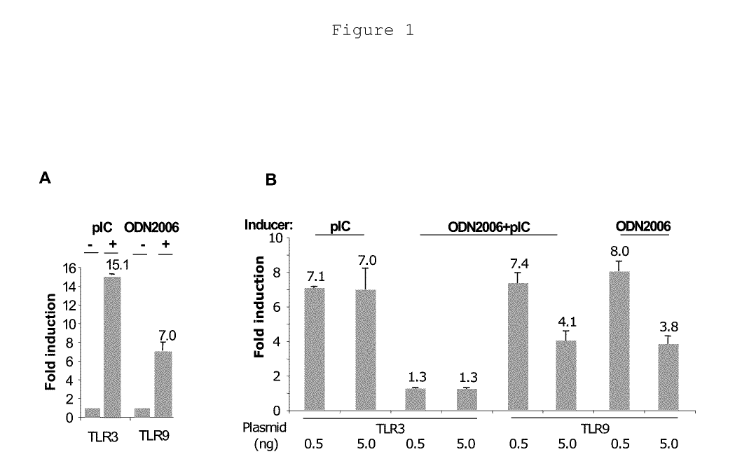

The activation of TLR3 requires its cognate ligand, an example of which is the double-stranded RNA mimic, poly(I:C), which can activate NF-κB reporter production by 4 to 16-fold above the uninduced control (FIG. 1A). The activation of TLR9 requires the addition of ODN2006 and is usually 3 to 8-fold above the uninduced control (FIG. 1A). ODN2006 contains a phosphorothioate backbone and CpG motifs and has the sequence shown in SEQ ID NO: 2.^{19,20} Phosphorothioates are known to increase the stability of the molecule in cells.²¹

The results shown in FIG. 1 indicate that ODN2006 inhibited poly(I:C)-induced TLR3 mediated activation of NF-κB and had no effect on TLR9 activity. TLR3 but not TLR9 is inhibited in the presence of poly(I:C) and ODN2006. In FIG. 1A, plasmids that can express TLR3 or TLR9 were transfected into HEK293T cells along with reporter plasmids coding for firefly luciferase under the NF-κB promoter and Renilla luciferase expressed from the thymidine kinase promoter. After expression of the plasmids for 24 h the cells were induced with either poly(I:C) or ODN2006. The bars represent fold induction of TLR activity over the uninduced control and are depicted by the numbers above the bars. In FIG. 1B, a cell based assay was performed as described above and induced with poly(I:C), ODN2006 or both. Fold induction of TLR3 and TLR9 activity were plotted.

When poly(I:C) (2.5 μg/ml) and ODN2006 (2 μM) were added, TLR3 activity was induced 1.3 fold above background in comparison to a 7-fold induction by poly(I:C) alone (FIG. 1B). Furthermore, this inhibition of TLR3 induction was observed in cells transfected with two different concentrations of TLR3 expression plasmids. The combination of the two ligands did not affect TLR9 activity, indicating that the inhibitory effect was specific to TLR3 (FIG. 1B).

### EXAMPLE 2

### Effect of Other Nucleic Acids on Modulation of TLR3 Activity

Two plasmid DNAs and single-stranded RNAs consisting of poly(I), poly(C), and poly(U) for were tested for their effects on inhibiting poly(I:C)-induced TLR3 activity (Table 1). TLR3 activity was measured as in Example 1. Unlike ODN2006, these other forms of nucleic acids did not reduce TLR3 activity to below 73% (Table 1). These results demonstrate that the single-stranded ODN2006 contains feature(s) required to inhibit TLR3 activity.

TABLE 1
Summary of the results from double-stranded DNAs and
single-stranded RNAs that are unable to inhibit TLR3 activity.

| Stimulation | | | % TLR3 |
|---|---|---|---|
| (error) | Form | [Poly (I:C)] | Activity |
| None | — | none | 15 (Ave. |
| | | | of 5 expt) |
| None | — | 2.5 | 100 (7) |
| Plasmid A, 12.5 μg/ml | dsDNA | 2.5 | 114 (9) |
| plasmid A, 25 μg/ml | dsDNA | 2.5 | 100 (9) |
| Plasmid B, 12.5 μg/ml | dsDNA | 2.5 | 102 (3) |
| plasmid B, 25 μg/ml | dsDNA | 2.5 | 114 (17) |
| poly(I), 12.5 μg/ml | ssRNA | 2.5 | 78 (3) |
| poly(I) 25 μg/ml | ssRNA | 2.5 | 75 (9) |
| poly(C), 12.5 μg/ml | ssRNA | 2.5 | 91 (22) |
| poly(C), 25 μg/ml | ssRNA | 2.5 | 78 (13) |
| poly(U), 12.5 μg/ml | ssRNA | 2.5 | 110 (9) |
| poly(U), 25 μg/ml | ssRNA | 2.5 | 109 (4) |
| poly(IU), 12.5 μg/ml | Annealed dsRNA | 2.5 | 76 (3) |
| poly(IU), 25 μg/ml | Annealed dsRNA | 2.5 | 73 (5) |

### EXAMPLE 3

### Effects of ODN2006 and poly (I:C) Concentration and Time of Addition on TLR3 Modulatory Activity

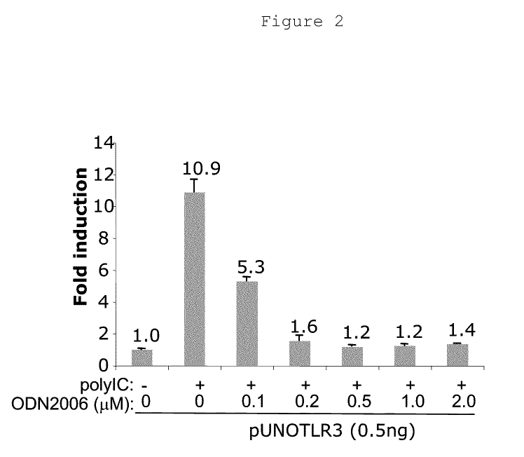

To examine whether the inhibition of TLR3 activity was dependent on ODN2006 concentration, ODN2006 was added to TLR3 activity assays to final concentrations of 0.1 to 2 μM (FIG. 2). The inhibitory effect was found to be dependent on ODN2006 concentration, with 50% inhibition being observed at ˜0.1 μM.

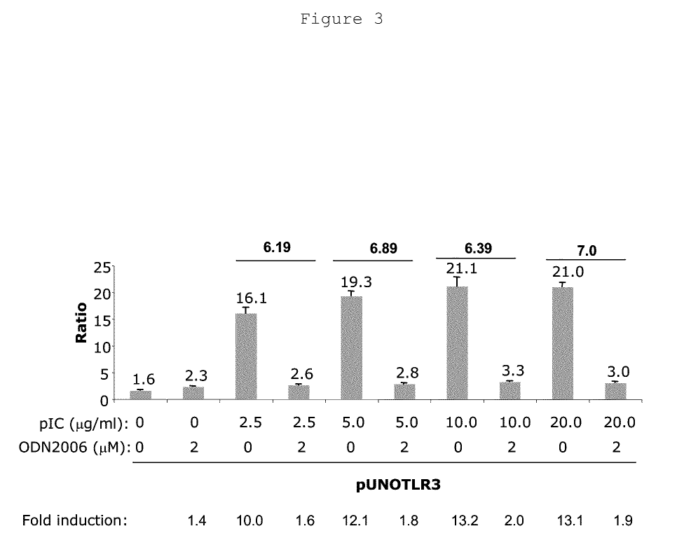

To determine whether ODN2006 mediated inhibition of TLR3 was affected by poly(I:C) concentration, poly(I:C) was added to the cells from 2.5 to 20 μg/ml while ODN2006 was kept constant at 2 μM (FIG. 3). After 24 h of expression different amounts of poly(I:C) from 2.5 to 20 μg/ml was added along with 2.0 μM ODN2006 and the ratio of firefly luciferase over Renilla luciferase is measured and plotted. TLR3 activity was measured as in Example 1. Fold induction of TLR3 activity over uninduced control is given at the bottom and the fold inhibition observed upon treatment with ODN2006 for each concentrations of poly(I:C) are given on the top of the graph. Since increasing poly(I:C) will affect the level of TLR3 activity even in the absence of ODN2006, the ratio of the inhibition by ODN2006 was calculated. The inhibitory ratio remained between 6.2 and 7.0-fold at all concentrations tested; higher poly(I:C) concentration did not apparently reverse the inhibition by ODN2006 (FIG. 3).

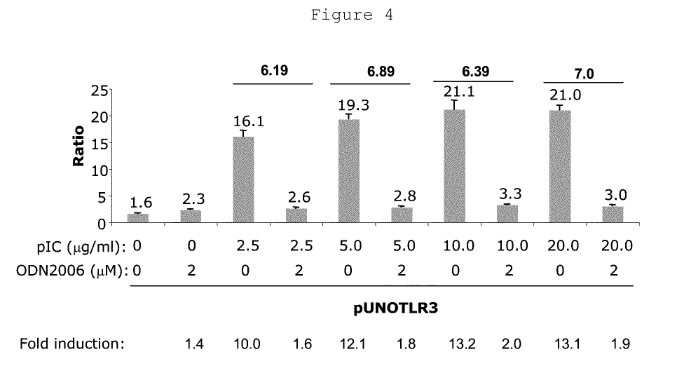

To analyze whether the effect of ODN2006 on TLR3 activation was dependent on the timing of poly(I:C) addition, 293T cells transfected to express TLR3 were either treated with poly(I:C) followed by ODN2006 addition 8 h later, or treated in the reverse order (FIG. 4). The results shown that the level of NF-κB activation was close to background when ODN2006 was added along with poly(I:C). However, when ODN was added 8 h after poly(I:C) treatment, 40% activity was observed. These results suggest that poly(I:C) could activate TLR3 until the addition of ODN2006. Furthermore, ODN2006 can inhibit TLR3 activity even after poly(I:C) had a chance to induce TLR3 activity.

While not wishing to be bound to any particular theory, it is thought that the results showing inhibition of TLR3 activity by ODN2006 in FIGS. 1-4 could be explained by three possible mechanisms: 1) ODN2006 has higher affinity to TLR3 than poly(I:C), 2) ODN2006 is competing for a factor, which could be an adapter for TLR3 or a common adapter for TLR3 and TLR9, or 3) ODN2006 could compete for a factor, which aids in transport of ligands from extracellular to intracellular areas.

### EXAMPLE 4

### Effect of ODN2006 on TLR3 Modulatory Activity in a TLR3 Mutant

A TLR3 mutant was expressed that was previously characterized to be dominant negative for wild-type TLR3 activity. A dominant negative version of TLR3 is inactive on its own, but when co-transfected with WT TLR3, the dominant negative can dimerize with WT TLR3 and reduce the activity of WT TLR3 by forming inactive complexes. The mutant TLR3ΔTIR, which has a deletion of the intracellular signaling domain, is documented to be a dominant negative mutant. TLR3ΔTIR inhibited TLR3 activity to 20% in the absence of ODN2006. In the presence of ODN2006, TLR3 inhibition was exacerbated, with only 6% of the activity. Two additional TLR3 mutants that also could not act as dominant negatives, a deletion of loop 1 and loop 2 (Table 2), were also inhibited by the presence of ODN2006 at 0.2 μM. These results indicate that ODN2006 can be used to inhibit TLR3 when it is present in a heterozygous form.

TABLE 2
Effects of ODN2006 on the activities of TLR3 mutants.

| | | ODN2006 | % TLR3 |
|---|---|---|---|
| WT TLR3 and: | Description | (μM) | Activ. (error) |
| pCDNA vector | plasmid vector | 0 | 100 (4) |
| pCDNA vector | ″ | 0.2 | 6 (2) |
| TLR3ΔTIR | TLR3 lacking intracellular | 0 | 20 (2) |
| | signaling domain. | | |
| | Dominant negative | | |
| TLR3ΔTIR | TLR3 lacking intracellular | 0.2 | 6 (1) |
| | signaling domain. | | |
| | Dominant negative | | |
| TLR3Δloop1 | TLR3 lacking residues | 0 | 98 (15) |
| | 335 to 343. | | |
| TLR3Δloop1 | TLR3 lacking residues | 0.2 | 7 (1) |
| | 335 to 343. | | |
| TLR3ΔLoop2 | TLR3 lacking residues | 0 | 79 (8) |
| | 547 to 554. | | |
| TLR3ΔLoop2 | TLR3 lacking residues | 0.2 | 8 (2) |
| | 547 to 554. | | |

The effect of the expression of other TLRs along with TLR3 on inhibition of TLR3 activity by ODN2006 addition was also examined. TLR1 through TLR8 were co-transfected at an equal molar ratio with TLR3 into 293T cells and poly(I:C) and ODN2006 at 0.2 μM were added to the cells and TLR3 activity determined as described above. The results indicated that ODN2006 was able to inhibit poly(I:C) mediated activation of TLR3 to background level in the presence of all other TLRs (Table 3). These results demonstrate that ODN2006 can inhibit TLR3 activity in the presence of TLRs 1 to 8.

TABLE 3
Expression of other TLRs cannot reverse ODN2006's
inhibitory activity on poly(I:C)-induced TLR3 activity.

| Vector | | | % TLR3 |
|---|---|---|---|
| expressing: | poly(IC) (μg/ml) | ODN2006 (μM) | Activ. (error) |
| φ | 0 | 0 | 14 |
| φ | 2.5 | 0 | 100 (6) |
| φ | 2.5 | 0.2 | 20 (3) |
| TLR1 | 2.5 | 0.2 | 18 (3) |
| TLR2 | 2.5 | 0.2 | 15 (1) |
| TLR3 | 2.5 | 0.2 | 23 (1) |
| TLR4 | 2.5 | 0.2 | 14 (1) |
| TLR5 | 2.5 | 0.2 | 15 (1) |
| TLR6 | 2.5 | 0.2 | 17 (1) |
| TLR7 | 2.5 | 0.2 | 18 (1) |
| TLR8 | 2.5 | 0.2 | 17 (1) |

### EXAMPLE 5

### Specificity of ODN2006 Activity

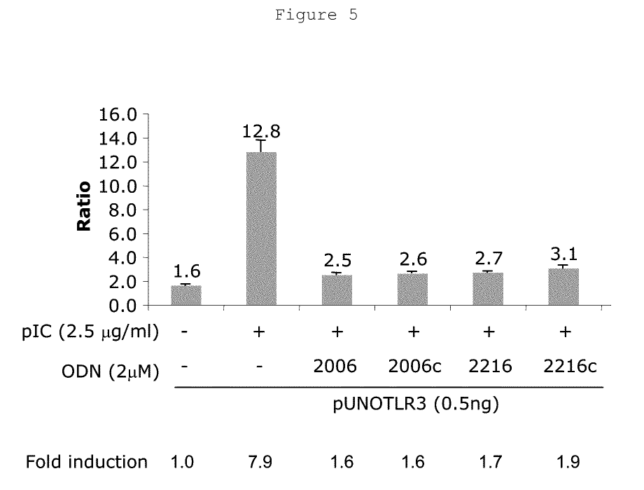

Several single-stranded deoxyoligonucleotides that cannot activate TLR9 activity as well as other activators of TLR9 were tested for their TLR3 inhibitory activity (FIG. 5). ODN2006c (SEQ ID NO: 3) is a variant of ODN2006 with an internal CpG nucleotide substituted by a GpC, a change associated with a loss of the ability to activate TLR9. ODN2216 (SEQ ID NO: 4) is a type A human TLR9 ligand while variant ODN2216c (SEQ ID NO: 5) contains a base substitution that renders ODN2216 to be a non-functional ligand of TLR9. TLR3 activity is depicted as the ratio of firefly luciferase over Renilla luciferase. The results show that all four nucleic acids inhibited TLR3 to similar degrees, suggesting that the inhibition is not specific to CpG sequence and that the ability to inhibit TLR3 is not related to the ability to activate TLR9. These results suggest that other DNA sequences and structures could be inhibitory to TLR3 activity.

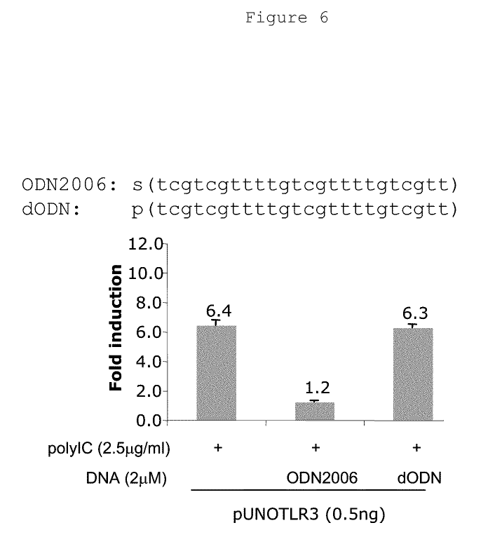

ODN2216 and ODN2216c have, respectively, one and five phosphorothioate bonds substituted for phosphodiester bonds at the 5′ and 3′ ends of the molecule, respectively. Since ODN2216 and ODN2216c are both potent inhibitors of TLR3, the number of phosphorothioate bonds can be reduced and TLR3 inhibition retained. Accordingly, a phosphodiester version of ODN2006 (with an identical base sequence as ODN2006) named dODN2006 was tested. dODN2006 was unable to inhibit TLR3 (FIG. 6). Other variants derived from dODN2006 were also unable to inhibit TLR3 activity (data not shown).

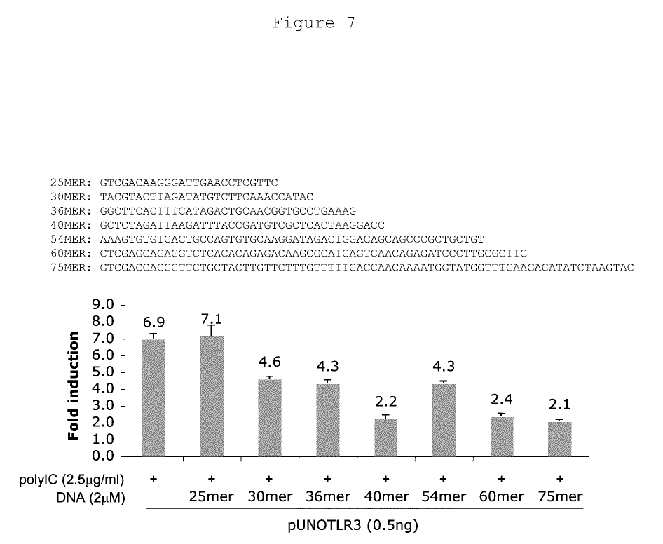

While not wishing to be bound to any theory, it is thought that phosphorothioates within ODN2006 likely decreased the rate of degradation enabling inhibition of TLR3 activity. It is expected that other single-stranded oligodeoxynucleotides that are inherently more stable to degradation due to their secondary or tertiary structures would cause some inhibition of TLR3. To test this hypothesis, a panel of seven deoxyoligonucleotides varying in sequence and length of 25 to 75-nt (FIG. 7) (SEQ ID NOs: 6-12) were randomly selected. When examined for TLR3 inhibition at 2 μM, a range of inhibitory activity was observed. Interestingly, there is a general trend between the degree of inhibitory activity and the length of the deoxyoligonucleotide, with the deoxyoligonucleotide of 25-nt having no obvious inhibitory activity. It is noted that the phosphodiester version of ODN2006, which was also unable to inhibit TLR3, was 24-nt in length. These results show that deoxyoligonucleotides lacking phosphorothioates can be used to inhibit TLR3 activity. In concert with the data from the potent TLR3 inhibitor ODN2006, longer deoxyoligonucleotides that can better withstand degradation when they are placed within a cellular environment are expected to be better TLR3 inhibitors provided that they are of a minimal length.

### EXAMPLE 6

### Effect of Deoxyoligonucleotide Length on TLR3 Activity

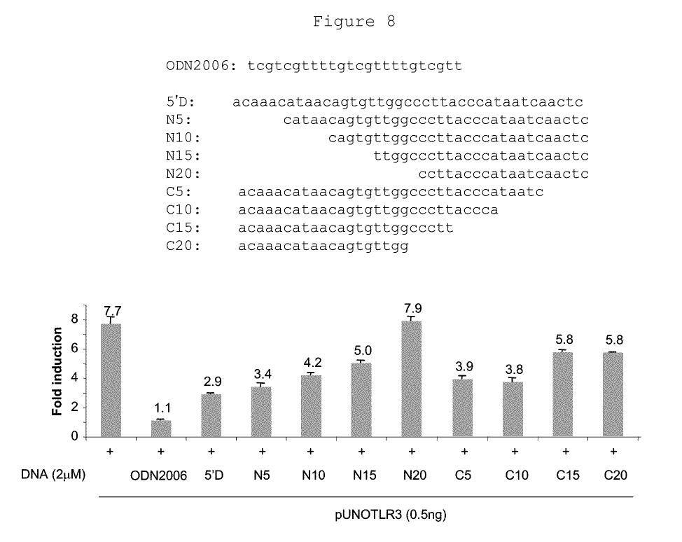

A 39-nt deoxyoligonucleotide with a phosphodiester backbone (5′D) (SEQ ID NO: 13) was selected as the prototype for further manipulations. Fold induction of TLR3 activity over uninduced control was plotted and the results show that 5′D inhibited poly(I:C)-induced activation of TLR3 by 60% (FIG. 8). A series of increasingly longer truncations from the 5′ terminus of 5′D (SEQ ID NOs: 14-17) resulted in a gradual loss of inhibitory activity. Further, deletions of 15- or 20-nt from the 3′ terminus of 5′D (SEQ ID NOs: 20, 21) resulted in DNAs that are less potent inhibitors than those with deletions of 5- to 10-nt (SEQ ID NOs: 18, 19). These results demonstrate that the length of the deoxyoligonucleotide is a factor in regulating the degree of inhibition of TLR3 activity.

### EXAMPLE 7

### Effect of Deoxyoligonucleotide Sequence on TLR3 Activity

To determine the effect of deoxyoligonucleotide base sequence on TLR3 inhibition, additions of six nucleotides to either termini of dODN2006 (phosphodiester backbone) that could form hairpin structures and potentially reduce sensitivity to nucleases were made in construct HP1 (Table 4) (SEQ ID NO: 22). When tested for effects on TLR3 activity, HP1 reduced TLR3 activity to 35%. This is a notable improvement from dODN2006, which was not inhibitory to TLR3, but not as potent as ODN2006 that contains phosphorothioates. Using HP1 as a platform, ODNs HP2 (SEQ ID NO: 23) and HP3 (SEQ ID NO: 24) were constructed where the loop sequence was replaced with a polyT or a polyA tract. These molecules were unable to inhibit TLR3 activity. In fact, a deoxyoligonucleotide containing the polyA tract was mildly stimulatory for TLR3 activity.

TABLE 4

The base sequence of a deoxyoligonucleotide can
contributes to its inhibitory activity.

| | | | |
|---|---|---|---|
| Potential | | | % TLR3 |
| inhibitor | | polylC | Activity |
| (2 μM) | Sequence | (μg/ml) | (error) |

| | | | |
|---|---|---|---|
| None | | 0 | 20 |
| None | | 2.5 | 100 (2) |
| ODN2006 | tcgtcgttttgtcgttttgtcgtt | 2.5 | 22 (2) |
| HP1 | CCGCCCtcgtcgttttgtcgttttgtcgttGGGCGG | 2.5 | 35 (1) |
| HP2 | CCGCCCttttttttttttttttttttGGGCGG | 2.5 | 80 (2) |
| HP3 | CCGCCCaaaaaaaaaaaaaaaaaaaaGGGCGG | 2.5 | 110 (6) |

These results indicate that the base sequence does contribute, either directly (perhaps by binding to a protein) or indirectly (perhaps by affecting degradation) to the inhibition of TLR3 activity. Based on the properties of the deoxyoligonucleotides examined, several sequences can inhibit TLR3 activity, although to varying degree. The observations with HP1 and its derivative suggest that the base sequence of an iOGN, as well as its length (FIG. 7) will be useful as platforms to design iOGN that can have varying potency in inhibiting TLR3 activity. This is advantageous since different medical conditions could require different degrees of cytokine modulation that can be achieved by varying the properties and/or concentrations of the iOGN.

### EXAMPLE 8

### Effect of Deoxyoligonucleotides on Cytokine Production in Human PBMC

To isolate human peripheral blood mononuclear cells (PBMC), whole blood was collected from human donors into heparin-coated syringes or heparin collection tubes. Approximately 50 ml of sterile Hank's Balanced Salt Solution (HBSS) (Invitrogen, Carlsbad, Calif.) was added to every 100 ml of blood. Thirty-eight ml of blood:HBSS was added to a 50 ml conical, and 11 ml Ficoll-Paque Plus solution (GE Amersham, Piscataway N.J.) was slowly layered underneath. The tubes were centrifuged at 400×g for 40 min. at room temperature. The centrifuge brake was turned off to preserve the gradient. The PBMC form a white layer just above the Ficoll. The PBMC from one conical were aspirated with a pipette into a new 50 ml conical. The tube was filled with HBSS to wash away the remainder of the Ficoll. The cells were spun at 600×g for 10 min. The cells were washed twice more with HBSS. After the final wash the pellet was resuspended in complete media: RPMI 1640 media/10% FBS/1× non-essential amino acids/1× sodium pyruvate) gentamycin. Gentamycin was purchased from Sigma; the other media components were purchased from Invitrogen. An aliquot of the cells was removed and mixed with 50 μg/ml Trypan blue to obtain a live cell count. The cells were plated in 48-well plates at a concentration of 3×10⁶ cells/well (0.5 mL/well).

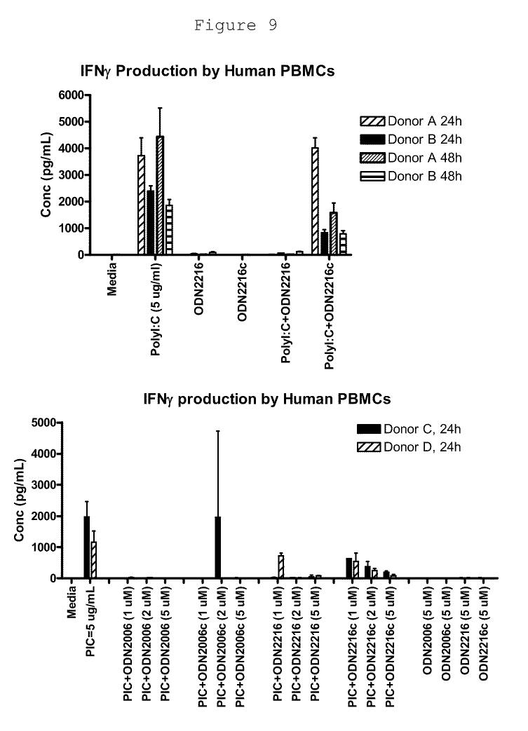

PBMC were collected from four unrelated donors (A, B, C and D) as described above. When treated with poly(I:C) at 5 μg/ml, the production of the cytokines IFNγ, IL-1β, IL-6, IL-12, IP-10, and MIG was measured using Luminex technology. Among the four donors, IFNγ levels were detected at approximately 1200 to 4000 pg/mL (FIG. 9). The levels of IL-12, IL-1β, IL6, IP-10 and MIG were all within the expected ranges (Table 5). These results confirm that the PBMCs are responding appropriately, although with a range that is to be expected due to difference in individuals.

PBMC were incubated with 5 μM (for Donor A) or 10 μM (for Donor B) ODN2216, ODN2006, ODN2216 control (ODN2216c) or ODN2006 control (ODN2006c) (synthesized by Invitrogen, Carlsbad, Calif., or purchased from Invivogen, San Diego, Calif.). The ssDNAs were used at 1, 2, or 5 μM for the experiment with Donor C and Donor D. The sequence of ODN2216 is 5′-ggG GGA CGA TCG TCg ggg gg-3′ (SEQ ID NO: 4). The sequence of ODN2216c control is 5′-ggG GGA GCA TGC TGg ggg gc-3′ (SEQ ID NO: 5). The sequence of ODN2006 is 5′-tcg tcg ttt tgt cgt ttt gtc gtt-3′ (SEQ ID NO: 2). The sequence of ODN2006c control is 5′-tgc tgc ttt tgt gct ttt gtg ctt-3′ (SEQ ID NO: 3). The bases in capital letters have phosphodiester linkages while those in lowercase have phosphorothioate linkages. Poly(I:C) was purchased from GE Amersham, reconstituted in PBS while heating at 50° C., and used at 5 μg/mL. Supernatants were harvested after 24 h or 48 h and frozen at −20° C. To determine TLR3 activity, cytokine levels were measured using the Human 10-Cytokine Luminex kit purchased from Upstate (Charlottesville, Va.). In some experiments, cytokine levels were measured using a custom Human 14-plex kit purchased from Invitrogen (Carlsbad, Calif.). The results are shown in FIG. 9 and each bar represents the mean +/−1SEM of two measurements from a single culture well (donors A and B) or one measurement from each of two culture wells (donors D and C).

The results indicate that when ODN2006 was added to the PBMC at the same time as poly(I:C), the levels of poly(I:C)-induced IFNγ from three donors were reduced to background levels when compared to the cells treated with poly(I:C) alone. Further, the effects were not limited to ODN2006, as the other ODNs tested, ODN2006c, ODN2216 and ODN2216c all had comparable effects when added to the cells to a final concentration of 5 μM. Importantly, these results mirror those observed in 293T cells (FIG. 5) and suggests that the effects of ODNs observed with 293T cells is indicative of more complex, biologically-relevant systems.

In order to extend the examination of the effects of ODNs, the production of several cytokines and chemokines by human PBMC were quantified. IL-12 and MIG production by PBMC from all four donors were reduced with ODN2216 or ODN2216c. Cells from three donors were tested with ODN2006 or ODN2006c, which also inhibited IL-12 and MIG production (Table 5). The effect of the ODNs on IP-10 was notable because three of the ODNs showed stronger inhibition than ODN2216. Together, these results demonstrate that ODNs can be designed to possess properties of selectively modulating one or more cytokine and/or chemokine production. Additional screening of the effects of cytokines and chemokines with ODNs of specific sequences and/or modifications could further improve the inhibitory effects.

TABLE 5
Single-stranded DNAs decrease poly(I:C)-induced IL-12, IL-
1β, IL-6, IP-10 and MIG production by human PBMCs (Donors A-D).

| | | | |
|---|---|---|---|
| A | B | C | D |

Levels of IL-12* (% Inhibition)

| | | | | |
|---|---|---|---|---|
| Media | 7 | 7 | 21 | 20 |
| CpG2216 | 7 | 7 | 32 | 35 |
| GpC ctrl for 2216 | 7 | 7 | 77 | 81 |
| CpG2006 | 7 | ND | 66 | 42 |
| GpC ctrl for 2006 | 7 | ND | 39 | 35 |
| Polyl:C | 422 | 444 | 1549 | 800 |
| Polyl:C + CpG2216 | 6.9 (100%) | 9.3 (99%) | 73 (97%) | 59 (95%) |
| Polyl:C + GpC ctrl for 2216 | 184 (57%) | 219 (51%) | 79 (96%) | 83 (92%) |
| Polyl:C + CpG2006 | 6.9 (100%) | ND | 76 (96%) | 49 (96%) |
| Polyl:C + GpC ctrl for 2006 | 6.9 (100%) | ND | 52 (98%) | 38 (98%) |

Levels of IL-1b (% Inhibition)

| | | | | |
|---|---|---|---|---|
| Media | 7 | 7 | 13 | 10 |
| CpG2216 | 17 | 7 | 19 | 15 |
| GpC ctrl for 2216 | 7 | 7 | 2627 | 853 |
| CpG2006 | 23 | ND | 14 | 16 |
| GpC ctrl for 2006 | 14 | ND | 12 | 13 |
| Polyl:C | 453 | 221 | 69 | 70 |
| Polyl:C + CpG2216 | 14 (98%) | 34 (88%) | 24 (81%) | 19 (85%) |
| Polyl:C + GpC ctrl for 2216 | 357 (21%) | 107 (53%) | 3687 | 2555 |
| Polyl:C + CpG2006 | 34 (94%) | ND | 15 (97%) | 12 (97%) |
| Polyl:C + GpC ctrl for 2006 | 28 (95%) | ND | 15 (97%) | 16 (90%) |

Levels of IL-6 (% Inhibition)

| | | | | |
|---|---|---|---|---|
| Media | 15 | 6.9 | 46 | 26 |
| CpG2216 | 972 | 609 | 2040 | 1078 |
| GpC ctrl for 2216 | 27 | 6.9 | 20000 | 20000 |
| CpG2006 | 466 | ND | 436 | 568 |
| GpC ctrl for 2006 | 209 | ND | 395 | 224 |
| Polyl:C | 1182 | 1343 | 3258 | 1523 |
| Polyl:C + CpG2216 | 786 (34%) | 985 (27%) | 2090 (36%) | 994 (35%) |
| Polyl:C + GpC ctrl for 2216 | 1634 | 969 (28%) | 20000 | 20000 |
| Polyl:C + CpG2006 | 541 (55%) | ND | 519 (85%) | 366 (77%) |
| Polyl:C + GpC ctrl for 2006 | 567 (53%) | ND | 947 (72%) | 517 (67%) |

| | |
|---|---|
| C | D |

Levels of IP-10 (% Inhibition)

| | | |
|---|---|---|
| Media | 80 | 48 |
| CpG2216 (5 uM) | 22823 | 23044 |
| GpC for 2216 (5 uM) | 12 | 15 |
| CpG2006 (5 uM) | 147 | 391 |
| GpC for 2006 (5 uM) | 71 | 72 |
| PIC = 5 ug/mL | 22031 | 24677 |
| PIC + CpG2216 (5 uM) | 14623 (34%) | 24154 |
| PIC + GpC for 2216 (5 uM) | 36 (100%) | 36 (100%) |
| PIC + CpG2006 (5 uM) | 998 (96%) | 1463 (94%) |
| PIC + GpC for 2006 (5 uM) | 2576 (89%) | 2958 (88%) |

Levels of MIG (% Inhibition)

| | | |
|---|---|---|
| Media | 54 | 51 |
| CpG2216 (5 uM) | 45 | 52 |
| GpC for 2216 (5 uM) | 61 | 67 |
| CpG2006 (5 uM) | 45 | 47 |
| GpC for 2006 (5 uM) | 48 | 49 |
| PIC = 5 ug/mL | 1212 | 2976 |
| PIC + CpG2216 (5 uM) | 68 (99%) | 189 (95%) |
| PIC + GpC for 2216 (5 uM) | 70 (99%) | 72 (99%) |
| PIC + CpG2006 (5 uM) | 47 (100%) | 49 (100%) |
| PIC + GpC for 2006 (5 uM) | 61 (99%) | 61 (100%) |

The present invention now being fully described, it will be apparent to one of ordinary skill in the art that many changes and modifications can be made thereto without departing from the spirit or scope of the appended claims.

1. A method for down modulating Toll-like Receptor 3 (TLR3) activity in a mammal comprising administering at least one TLR3 inhibitory oligonucleotide (iOGN) to the mammal.

2. The method of claim 1 wherein the iOGN is about 17 to about 75 nucleotides in length.

3. The method of claim 1 wherein the iOGN comprises modifications in the base, ribose, phosphodiester or phosphorothioate groups.

4. The method of claim 1 wherein the iOGN has the sequence shown in SEQ ID NO: 2, 3, 4, 5, 7, 8, 9, 10, 11, 12, 13, 14, 15, 16, 18, 19, 20, 21, 22 or 23.

5. The method of claim 1 wherein the mammal is a human.

6. The method of claim 1 wherein the iOGN is conjugated to a monoclonal antibody, antibody fragment, alternative scaffold, protein, or peptide specific for TLR3.

7. The method of claim 1 further comprising administering the at least one iOGN in combination with another non-iOGN modulator of TLR3 activity.

8. The method of claim 7 wherein the non-iOGN modulator is an antibody, MIMETIBODY™ construct, or small molecule specific for TLR3 or another TLR receptor.

9. The method of claim 7 wherein the non-iOGN modulator is an antibody, MIMETIBODY™ construct, or small molecule specific for a ligand for TLR3 or another TLR receptor.

10. The method of claim 1 further comprising administering the at least one iOGN in combination with an anti-inflammatory agent.

11. The method of claim 1 further comprising administering the at least one iOGN in combination with an anti-microbial agent.

12. The method of claim 1 further comprising administering the at least one iOGN in combination with an anti-viral agent.

13. A method of treating or preventing an inflammatory condition comprising administering a therapeutically effective amount of a TLR3 iOGN to a patient in need thereof for a time sufficient to treat or prevent the inflammatory condition.

14. The method of claim 13 wherein the inflammatory condition is infection-associated.

15. The method of claim 13 wherein the inflammatory condition is pancreatitis, alopecia areata, atopic dermatitis, autoimmune hepatitis, Bechet's disease, cirrhosis, hepatic fibrosis, Crohn's disease, regional enteritis, inflammatory vitilgo, multiple sclerosis, pemphigus/pemphigoid, primary biliary cirrhosis, psoriasis, scleroderma, sclerosing cholangitis, systemic lupus erythematosus, lupus nephritis, toxic epidermal necrolysis, ulcerative colitis, warts, hypertrophic scarring, keloids or acetaminophen-induced injury.

16. A method of treating or preventing an necrotic condition comprising administering a therapeutically effective amount of a TLR3 iOGN to a patient in need thereof for a time sufficient to treat or prevent the necrotic condition.

17. The method of claim 16 wherein the necrotic condition is acute renal failure.

18. A method of treating or preventing an infectious disease comprising administering a therapeutically effective amount of a TLR3 iOGN to a patient in need thereof for a time sufficient to treat or prevent the infectious disease.

19. The method of claim 18 wherein the infectious disease is anthrax, C. Difficile infection, encephalitis/meningitis, endocarditis, Hepatitis C, Influenza/severe acute respiratory syndrome (SARS), pneumonia, sepsis, burn or trauma-related skin conditions or systemic inflammatory response syndrome (SIRS).

20. A method of treating or preventing a cardiovascular disease comprising administering a therapeutically effective amount of a TLR3 iOGN to a patient in need thereof for a time sufficient to treat or prevent the cardiovascular disease.

21. The method of claim 21 wherein the cardiovascular disease is atherosclerosis, myocardial infarction or stroke.

22. A method of treating or preventing type I or type II diabetes comprising administering a therapeutically effective amount of a TLR3 iOGN to a patient in need thereof for a time sufficient to treat or prevent the type I or type II diabetes.

23. A method of treating or preventing cancer comprising administering a therapeutically effective amount of a TLR3 iOGN to a patient in need thereof for a time sufficient to treat or prevent the cancer.

24. The method of claim 23 wherein the cancer is acute leukemia, breast cancer, chronic leukemia, colorectal cancer, esophageal cancer, gastric cancer, Hodgkins disease, lung cancer, lymphoma, melanoma, multiple myeloma, Non-hodgkin's disease, ovarian cancer, pancreatic cancer, prostrate cancer, sarcoma, renal cell cancer, head and neck cancers or virally-induced cancers.

25. A method of treating or preventing rheumatoid disease comprising administering a therapeutically effective amount of a TLR3 iOGN to a patient in need thereof for a time sufficient to treat or prevent the rheumatoid disease.

26. The method of claim 25 wherein the rheumatoid disease is autoimmune thyroiditis, autoimmune vasculitis, disoid lupus erythematosus, lupus nephritis, osteoarthritis, polychondritis, polymyalgia rheumatica, psoriatic arthritis, rheumatoid arthritis, systemic lupus erythematosus or systemic scleroderma.

27. A method of treating or preventing pulmonary disease comprising administering a therapeutically effective amount of a TLR3 iOGN to a patient in need thereof for a time sufficient to treat or prevent the pulmonary disease.

28. The method of claim 27 wherein the pulmonary disease is acute lung injury, acute respiratory distress syndrome, acute asthma exacerbations, acute COPD exacerbations, idiopathic pulmonary fibrosis or sarcoid.

29. A method of treating or preventing neurological disorders comprising administering a therapeutically effective amount of a TLR3 iOGN to a patient in need thereof for a time sufficient to treat or prevent the neurological disorder.

30. The method of claim 29 wherein the neurological disorder is stroke, Alzheimer's disease, meningitis, spinal cord injury, trauma, demyelination disorders or pain.

31. An iOGN having the sequence shown in SEQ ID NO: 7, 8, 9, 10, 11, 12, 13, 14, 15, 16, 18, 19, 20, 21, 22 or 23.
